(19) United States
(12) Patent Application Publication
(10) Pub. No.: US 2026/0102065 A1
(43) Pub. Date: Apr. 16, 2026

(54) System And A Method For Measuring Functional Characteristics Of Mammalian Retina

(71) Applicant: Quantal Vision Technologies Oy, Espoo (FI)

(72) Inventors: Petri Ala-Laurila, Espoo (FI); Gabriel Peinado, Helsinki (FI)

(73) Assignee: Quantal Vision Technologies Oy, Espoo (FI)

(21) Appl. No.: 19/264,009

(22) Filed: Jul. 9, 2025

(30) Foreign Application Priority Data
Oct. 10, 2024 (FI) FI20247148

(51) Int. Cl. A61B 3/14

(52) U.S. Cl. CPC A61B 3/14

(57) ABSTRACT

The invention concerns systems and methods for measuring certain characteristics of eyes of mammals. Specifically, the invention concerns systems and methods for measuring functional characteristics of the mammalian retina noninvasively. In an embodiment a system for determining a characteristic of mammalian retina is provided. The system has a light source for providing stimuli to the eye and an arrangement for monitoring responses of the eye to the stimulus. In an embodiment the light source is arranged to provide stimuli using light within a first frequency range at an intensity causing on average less than 100 isomerizations per second per rod cell of the retina, the average being calculated over at least one stimulus cycle; the arrangement for monitoring responses of the eye comprises at least a camera for imaging the eye and an imaging light source for illuminating the eye; said infrared light source being arranged to provide light within a second frequency range and at an intensity causing an isomerization rate lower than said light stimulus, said system further comprising a control unit arranged to control the light source providing stimuli to the eye, receive images from said camera, and determine response of the pupil to the stimulus from said received images.

### FIELD OF THE INVENTION

The invention concerns systems and methods for measuring certain characteristics of the eyes of mammals. Specifically, the invention concerns systems and methods for measuring functional characteristics of the mammalian retina.

### DESCRIPTION OF PRIOR ART

Studies of vision can provide a wealth of information about neural functions. Vision starts as light enters the eye, gets captured by the photoreceptor cells and processed in the neural circuits of the retina—an accessible part of the brain. The neural circuits of the human retina have well-defined outputs originating from over 30 different retinal ganglion cell (RGC) types that convey visual information as action potentials to the brain via the optic nerve.

Understanding how retinal action potentials drive visual perception has been an extremely difficult problem to resolve. This is due to the difficulty of accessing the relevant neural signals with sufficient resolution and due to the complexity of visually-guided behavior and due to the lack of noninvasive tools to assess retinal cell-type specific function. Unraveling the link between perception and retinal function would not only help to understand the neural basis of visual perception but also allow to assess the mechanistic basis of visual impairments.

Vision under extremely dim light conditions offers great promise for resolving many of the key challenges that currently hinder bridging visual perception to retinal function. At the dimmest conditions of light illumination perceivable to humans, less than hundred photons impinge into the cornea of the eye, causing a handful of photoisomerization events (Hecht S., Shlaer S., Pirenne M. H. (1942) ENERGY, QUANTA, AND VISION, J Gen Physiol 25 (6): 819-840). A photoisomerization is a process where a photon is captured by a visual pigment molecule in a retinal photoreceptor cell, causing a conformational change and an activation of the molecule. This activation leads to the generation of an electric signal that is transmitted to the brain via the neural circuitry of the retina. In this specification, we use the term isomerization as a shorthand for photoisomerization, and we refer to the capture of photons by rhodopsin, the visual pigment in rod photoreceptor cells. An isomerization event (denoted as R*) in a rod photoreceptor is the basic input to neural processes in the retina, the basic event where incoming light is converted into an electric signal in a rod photoreceptor cell processable by the neural circuitry of the retina. A review of some of the challenges and current knowledge about human vision in dim light is presented in the book chapter by Kiani, R., Ala-Laurila, P., and Rieke, F. (2020). 1.16 “Seeing With a Few Photons: Bridging Cellular and Circuit Mechanisms With Perception”, The Senses: A Comprehensive Reference (Second Edition), Fritzsch, B., ed. (Oxford: Elsevier), pp. 293-308.

Some key benefits on studying vision under extremely dim light conditions are the following.

First, dark-adapted humans can detect only a handful of photons showing that behavioral performance comes extremely close to the fundamental limits of physics. In this way, application of physical laws provides quantitative predictions against which to compare empirical measurements.

Second, contrary to daylight conditions, where retinal processing involves many neural circuits and RGC types, all visual signals originating from a small number of photons at the dimmest light levels are confined to a well-defined retinal circuit shared among all mammals—the rod bipolar pathway. Only the most sensitive retinal RGC types receive input from this retinal pathway and can be split into two distinct classes: ON RGCs responding to light increments by increasing their firing rate and OFF RGCs responding to light increments by decreasing their firing rate.

Third, the most sensitive ON and OFF RGC types are readily identifiable and exhibit a striking similarity across mice, monkeys and humans in their response properties. However, methods for examining these neural pathways under extremely dim light conditions are very cumbersome and slow, limiting their use to basic scientific research. Typical observations utilize conscious vision, requiring test subjects to respond whether they can see a stimulus or not. This is a slow and demanding task for humans, requiring instructing the person before testing and a careful consideration for subjective biases. Faster methods are needed, as well as methods that are amenable to automation and building into testing systems.

### SUMMARY OF THE INVENTION

The invention provides a system and a method for measuring noninvasively functional characteristics of the mammalian retina.

The inventors have had the insight that pupillary responses in mammals to various stimuli can be reliably detected at such extremely dim levels of retinal stimuli that only the most sensitive signal pathways of the retina continue to function. Further, the inventors have found that these most sensitive signal pathways are very sensitive to many types of retinal damage.

The inventors have developed a way to detect pupillary responses at such extremely dim stimulus intensities without the detection itself appreciably disturbing the causation of the pupillary responses to the stimulus.

The method developed by the inventors produces reliable results even at stimulus intensities far below an intensity causing one photon capture per second per rod cell of the retina.

In other words, the inventors have developed a method to use the pupil as a readout for extremely sensitive characterization of the retina, a method far more sensitive than any known prior art method.

In the method, the retina is stimulated using light stimulus within a first wavelength range with an intensity that causes an isomerization rate of less than 100 R* per rod per second. Calculations for stimuli ranging from 0.0001 to 100 R* per rod per second produced by various wavelengths are outlined later in this specification for examples. The pupillary light responses are detected by illuminating the pupil using light within a second wavelength range, with such a wavelength and intensity which causes an isomerization rate lower than the intensity of the retinal stimulus. Imaging of the pupil contractions is accomplished by a camera sensitive to light in the second wavelength range.

In an embodiment of the invention the first wavelength range is from 300 nm to 900 nm.

In an embodiment of the invention the pupil may be illuminated for imaging purposes with light having a wavelength of 700 nm or more, i.e. the second wavelength range is in this particular embodiment 700 nm or longer wavelengths. In a further embodiment of the invention, the second wavelength range is wavelengths of 850 nm or longer.

In an embodiment of the invention the intensity and wavelength of the imaging light within the second wavelength range may be chosen so as to cause at least one order of magnitude lower isomerization rate than the stimulus.

In a further embodiment of the invention the intensity and wavelength of the imaging light within the second wavelength range may be chosen so as to cause an isomerization rate at or below the level of noise originating from spontaneous isomerizations of rod photoreceptor visual pigments in the dark, which according to literature is roughly ˜0.01 R* per rod per second, Field et al. (2005), Retinal processing near absolute threshold: from behavior to mechanism. Ann. Rev. Physiol. 67, 491-514.

In a further embodiment of the invention, pupillary responses to positive stimulus are monitored and the smallest positive stimulus levels that result in detectable pupillary responses are determined, and a characteristic of the retina is determined from said determined smallest positive stimulus levels. In such an embodiment, a positive stimulus is a stimulus where a constant level of illumination is increased for a period of time.

In a further embodiment of the invention, pupillary responses to negative stimulus are monitored and the smallest negative stimulus levels that result in detectable pupillary responses are determined, and a characteristic of the retina is determined from said determined smallest negative stimulus levels. In such an embodiment a negative stimulus is a stimulus where a constant level of illumination is reduced for a period of time.

### BRIEF DESCRIPTION OF THE DRAWINGS

Various embodiments of the invention will be described in detail below, by way of example only, with reference to the accompanying drawings, of which

FIG. 1 illustrates a system for determining a characteristic of mammalian retina according to an embodiment of the invention,

FIGS. 2A and 2B illustrate various methods according to embodiments of the invention, and

FIG. 3 illustrates a graph of the sensitivity of rod visual pigment, rhodopsin.

### DETAILED DESCRIPTION OF CERTAIN EMBODIMENTS

The following embodiments are exemplary. Although the specification may refer to “an”, “one”, or “some” embodiment(s), this does not necessarily mean that each such reference is to the same embodiment(s), or that the feature only applies to a single embodiment. Features of different embodiments may be combined to provide further embodiments.

In the following, features of the invention will be described with a simple example of system for determining a characteristic of mammalian retina with which various embodiments of the invention may be implemented. Only elements relevant for illustrating the embodiments are described in detail. Details that are generally known to a person skilled in the art may not be specifically described herein.

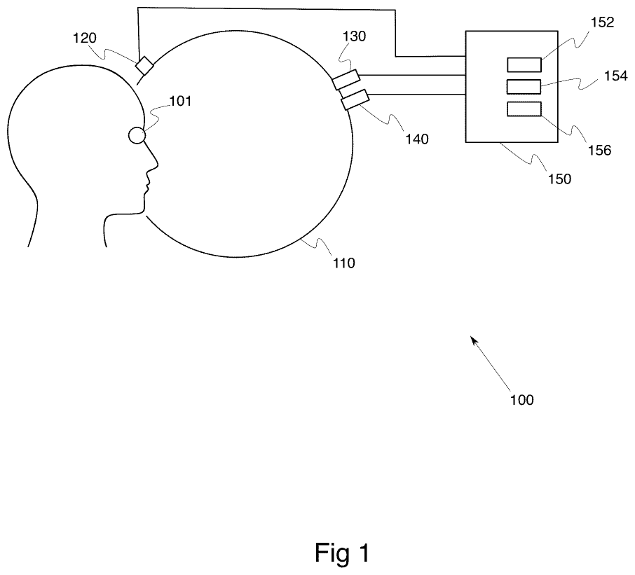

FIG. 1 illustrates a system 100 for determining a characteristic of mammalian retina according to an embodiment of the invention.

FIG. 1 shows a stimulus creation arrangement arranged to create light for reception by the eye 101 of the individual being examined by the system. In the embodiment illustrated in FIG. 1 the stimulus creation arrangement comprises a spherical projection chamber 110 and a light source 120. The light source 120 projects light within a first wavelength range to the inside surface of the chamber 110, which is in the field of view of the eye 101 of the individual being examined.

The arrangement illustrated in FIG. 1 has the advantage that it is able to provide stimulus over the whole field of view of the eye 101. However, the light source 120 can in various embodiments of the invention also be configured to project light in one or more patterns of different shapes and/or sizes. An embodiment in which the light source 120 projects light to an area which corresponds to a smaller than full field of view of the eye 101 has the advantage that in such an embodiment, stimuli that are localized to only a certain area of the retina can be provided.

In an embodiment of the invention the intensity of the light provided by the light source 120 is adjustable. In a further embodiment of the invention, the intensity of the light provided by the light source 120 can be controlled by a control unit 150 of the system. FIG. 1 illustrates that the light source 120 is connected to the control unit 150. In such an embodiment, the control unit 150 may direct the light source 120 to emit light in order to create a stimulus at a desired intensity and vary the intensity in different stimuli. Further, the control unit may adjust the light intensity to zero between stimuli, or to a predefined intensity level between different stimuli. Further, the control unit can control the light intensity to provide stimuli with various lengths of time.

In an embodiment of the invention the light source 120 comprises one or more light emitting diodes (LEDs). However, the invention is not limited to use of LEDs as light generating devices, as many different arrangements known to a man skilled in the art can be used in different embodiments of the invention. For example, in a further embodiment of the invention a DLP (digital light processing) projector is used in combination with a spherical mirror in order to project structured patterns of illumination into the sphere.

In an embodiment of the invention the system 100 comprises more than one light source 120 projecting light to the inside of the chamber 110. With such a structure, provision of localized stimuli and/or providing an even lighting to the whole inside of the chamber 110 may be easier than with only one light source 120.

In further embodiments of the invention other structures than the chamber 110 and a light source installed in the chamber can be used to provide light stimulus. For example, in an embodiment of the invention a headset worn on the head of the test subject can be arranged to provide light stimulus and to image the pupil with imaging light in the second wavelength range.

In a further embodiment of the invention the first wavelength range of light provided by the stimulus creation arrangement is roughly around 500 nm, for example between 450 nm and 520 nm. In a further embodiment of the invention, the first wavelength range is between 450 nm and 600 nm. In a still further embodiment of the invention, the first wavelength range is between 450 nm and 700 nm. In a still further embodiment of the invention, the first wavelength range is between 450 nm and 800 nm. In a still further embodiment of the invention, the first wavelength range is between 300 nm and 900 nm.

In an embodiment of the invention, the light source 120 is arranged to emit light at substantially a single wavelength or a very narrow band of wavelengths such as provided by a single light emitting diode.

In a further embodiment of the invention, the light source 120 is arranged to emit light at a plurality of wavelengths within the first wavelength range. In such an embodiment, the light source 120 can comprise a plurality of LEDs, each emitting light at its own wavelength.

In a further embodiment of the invention the light source 120 is arranged to emit light on one or more wavelength bands within the first wavelength range.

FIG. 1 is merely an illustration of one exemplary embodiment showing a particular structure of a stimulus creation arrangement. In different embodiments, the chamber 110 can be of different shape than a spherical chamber, and the chamber 110 can be implemented in different sizes. The chamber 110 can be for example a so called Ganzfeld sphere, which is a known arrangement typically used in psychophysical studies of the human vision.

In a further embodiment of the invention, the stimulus creation arrangement can be implemented using a head-mounted headset. In a still further embodiment of the invention, the stimulus is created by direct projection into the eye.

In a still further embodiment of the invention in which the system 100 is used to determine a characteristic of the retina of a small mammal, the stimulus is created by projecting light to the inside surfaces of an experimental enclosure within which the small mammal being examined is placed. In a further embodiment of the invention, the system 100 comprises an arrangement for detecting the direction of the head of the mammal being examined. Such an arrangement can comprise for example a camera. In such an embodiment, the light projected to the surfaces of the experimental enclosure can be controlled at least in part using the information about the direction of the head, in order to keep the stimulus in the field of view of the mammal.

FIG. 1 also illustrates an eye imaging arrangement. In the example of FIG. 1, the eye imaging arrangement comprises an imaging light source 130 and a camera 140. In the example of FIG. 1, both the imaging light source 130 and camera 140 are connected to the control unit 150. In an embodiment of the invention, the control unit 150 is arranged to control the imaging light source 130 in order to control the intensity of the imaging light produced by the imaging light source 130. In an embodiment of the invention the control unit 150 is arranged to control the camera in order to initiate capture of one or more images by the camera. In an embodiment of the invention the control unit 150 is further arranged to read images produced by the camera.

In an embodiment of the invention the imaging light source 130 comprises one or more LEDs for producing light within the second wavelength range. However, the invention is not limited only to using LEDs for generation of imaging light, since in further embodiments other types of light sources such as incandescent bulbs can be used. A man skilled in the art knows that there are many other ways to generate light in the second wavelength range, whereby these are not discussed further in this specification.

In a further embodiment of the invention the imaging light source 130 further comprises a filter to reduce the intensity of visible light produced by the imaging light source, if any. A man skilled in the art knows different types of filters, such as interference filters, that can be used for this purpose, whereby these are not discussed any further in this specification.

In an embodiment of the invention, the second wavelength range is wavelengths of 850 nm or longer.

In a further embodiment of the invention, the second wavelength range is wavelengths of longer than 900 nm.

In an embodiment of the invention, the imaging light source 130 is arranged to produce imaging light on more than one wavelength within the second wavelength range. For example, the imaging light source can comprise more than one LED for provision of light at multiple different wavelengths. In such an embodiment, the control unit can adjust the wavelength by controlling which of the light sources such as leds are used for producing imaging light.

In the example of FIG. 1, the eye imaging arrangement comprises a camera 140 for imaging the eye under the lighting produced by the imaging light source 130. The camera is arranged to produce images of the eye and especially the pupil of the eye under control of the control unit 150.

In an embodiment of the invention the control unit 150 is arranged to control provision of stimulus using the stimulus creation arrangement, receive and store images produced by the camera 140 under lighting of the imaging light source 130, and determine based on received images whether the stimulus cause the pupil to change in any way.

In various embodiments of the invention the control unit can be arranged to determine the size of the pupil in each image and observe whether the determined size changes after provision of stimulus. The size of the pupil can be determined in various ways in different embodiments of the invention. For example, the size of the pupil can be determined by measuring the diameter of the pupil from an image. In a further embodiment of the invention, the size of the pupil is determined by measuring the area of the pupil from a received image. In a further embodiment of the invention, the size of the pupil is determined by measuring the circumference of the pupil from a received image.

In a further embodiment of the invention, the size of the pupil is determined by fitting a circle to the image of the pupil and using the parameters of the fitted circle as a measure of the size of the pupil in the image.

In a further embodiment of the invention, the size of the pupil is determined by fitting an ellipse to the image of the pupil and using the parameters of the fitted ellipse as a measure of the size of the pupil in the image.

In a typical image of a pupil, the interface between the pupil and the iris is not exactly sharp but rather a gradual change. Consequently, a man skilled in the art knows many different ways of determining a value for the size of the pupil from such digital images, whereby the invention is not limited to the ways enumerated in this specification.

In an embodiment of the invention the control unit is arranged to monitor changes in the pupil within a predetermined time after the start of a stimulus. Such an embodiment allows the inventive system to detect only relatively fast responses, which happen in the order of seconds. In an embodiment of the invention the control unit is arranged to monitor the pupil from 0 to 30 seconds after the start of a stimulus. In a further embodiment of the invention the control unit is arranged to monitor the pupil from 0 to 3 seconds after the start of a stimulus. In a still further embodiment of the invention the control unit is arranged to monitor the pupil from 0 to 1 seconds after the start of a stimulus.

FIG. 1 also illustrates a control unit 150. The control unit 150 can be implemented in various ways in different embodiments of the invention.

In an embodiment of the invention, the control unit 150 is a computing device having a processor 152 and a memory 154. The memory comprises software code executed by the processor, which causes the processor to control the system 100 in ways described in this specification.

In a further embodiment of the invention, the system 100 may comprise more than one computing device for carrying out the functions ascribed in this specification to the control unit 150. In a further embodiment of the invention, the control unit 150 also comprises a communication unit allowing the control unit 150 to communicate with one or more servers over a communications network.

FIG. 1 also illustrates a mass memory 156 such as a database, a disk drive or a mass storage unit of any other type for storing images received from the camera and any other data. FIG. 1 illustrates the mass memory 156 as being installed in the control unit 150. However, in different embodiments of the invention the mass memory 156 may be connected to another computing device or a server, whether local to the control unit 150 or a remote computing device connected to the control unit via a data communications network.

In an embodiment of the invention, the control unit 150 also comprises a display for presenting a user interface. In a further embodiment of the invention, the user interface for the control unit 150 is arranged at a remote computing device connected to the control unit 150 via a data communications network.

In an embodiment of the invention, the system 100 is arranged to provide full field stimulus, i.e. stimulus that covers all of the retina. In such an embodiment, the stimulus light source 120 provides light to at least such a large area on the inside surface of the chamber 110 that the full field of view of the eye 101 is covered.

In a further embodiment of the invention the system 100 is arranged to provide localized stimulus. In such an embodiment, the stimulus light source 120 provides light to a predetermined part or parts of the inside surface of the chamber 110 so that only a certain desired area or areas of the retina of the eye 101 receive the stimulus. Such an embodiment allows examination of desired localized regions of the retina.

In an embodiment a positive localized stimulus can be arranged as an increase in the light from the stimulus light source 120 into a specific area or areas inside the chamber 110 for a predetermined time period. The increase can be relative to a previous level of light, or zero light from the stimulus light source 120.

In an embodiment of the invention a negative localized stimulus can be arranged as a localized reduction in the light from the stimulus light source 120 in a specific area or areas inside the chamber 110 for a predetermined time period, i.e. reduction from a previous level of light from the stimulus light source 120. The reduction can be a part or all of the light from the stimulus light source 120 for the localized region.

Embodiments of the invention utilizing localized stimuli have the advantage of providing localized information about possible local malfunctions of the retina.

Embodiments of the invention providing stimulus over the full field of view of the eye have the advantage that pupillary responses can be detected as a response to stimulus having lower photon densities than in the case of local stimulus. Thus, using full field stimulus allows measurements that reflect functioning of the most sensitive neural pathways.

In an embodiment of the invention the system 100 is arranged to determine a threshold where a stimulus produces a detectable pupillary response. In such an embodiment, the system is arranged to provide a series of increasing stimulus, and to observe when pupillary responses begin to be detectable against the background noise (no stimulus condition).

The exact definition of when a response is detectable can be devised in many different ways by a man skilled in the art. There are many different approaches known to a man skilled in the art about how to interpret raw observations. For example, a simple definition of a threshold can be such, that a response is determined to be detected when more than 50% of stimulus of a given strength produce a change in the pupil as compared to no stimulus condition. The invention is not limited to any specific way of determining such a threshold.

A stimulus can be a negative stimulus, in which the stimulus is a reduction in the intensity of light provided to the retina. For providing a negative stimulus, a base level of light is provided, and a reduction of light from that level for a period of time is the negative stimulus. The negative stimulus can be a full field stimulus in which the light level in all of the field of view of the eye is reduced, or local in which case the light is reduced only within a certain area.

A positive stimulus is, conversely, an increase in the level of light provided to the retina. The increase can be from total darkness, or from a certain base level of light.

In an embodiment of the invention in which the threshold for negative stimulus and the threshold for positive stimulus are determined, the base level of light may be the same for both types of stimulus.

The characteristic determined using the system illustrated in FIG. 1 can be any measurement result provided by the system, or any result calculated or otherwise determined from one or more measurement results provided by the system.

The characteristic can be for example how the pupil responds to certain stimuli, which at the stimulus levels described in this specification is very dependent on the functioning of the most sensitive neural circuit of the retina (the rod bipolar pathway) and thus indicative of the condition of the retina. The characteristic can in further embodiment of the invention be a result determined at least in part of a measurement, such as the determination of the smallest negative or positive stimuli that provides a detectable pupillary response.

The invention is not limited to determination of any particular characteristic, as a man skilled in the art can use the inventive system and method with different stimulus levels and types to determine a desired characteristic.

In the following, we discuss calculations of isomerization rates in retinal rod cells based on intensity and wavelength.

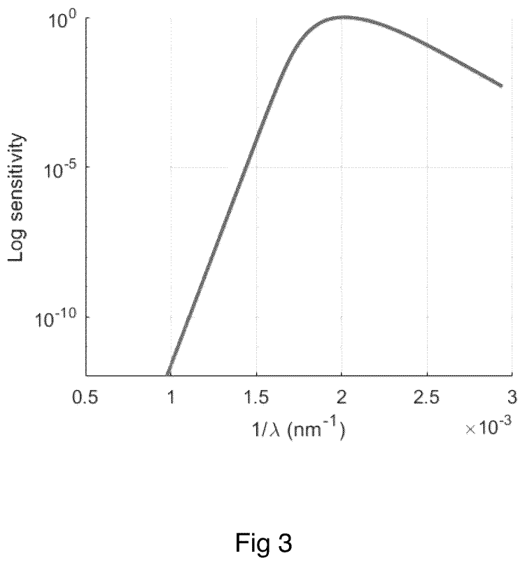

Not all photons impinging on a retinal rod cell cause isomerizations. A large contributing factor is the spectral sensitivity of rhodopsin. A graph of the sensitivity of rhodopsin is presented in FIG. 3. The vertical axis is the sensitivity on a logarithmic scale with the largest value being normalized to 1. The horizontal axis corresponds to inverse of wavelength. As can be seen from FIG. 3, the sensitivity of rhodopsin has a maximum around the wavelength of 500 nm (i.e. at 2*10³ nm⁻¹ in the horizontal scale of FIG. 3), and the sensitivity becomes lower at longer wavelengths. As the vertical axis of the graph of FIG. 3 is logarithmic, one can observe that the sensitivity is several orders of magnitude lower at the long wavelength end of the visible spectrum.

We note that although the sensitivity of rhodopsin is lower at longer wavelengths, isomerizations can and do still happen even at near infrared wavelengths, only with a much lower probability than around the peak sensitivity. As FIG. 3 shows, at a wavelength of 1000 nm (i.e. at 0.001 nm⁻¹ in the horizontal scale of FIG. 3) the probability is more than 10 orders of magnitude lower than at the peak.

In an embodiment of the invention, the stimulus is given as light at or near the top of the rhodopsin sensitivity graph, i.e. at 500 nm or within the range from 400 nm to 600 nm, and the imaging of the pupil is performed using imaging lighting within the second wavelength range. Such an embodiment has the advantage that with such arrangement of wavelengths, the imaging lighting is performed at a wavelength where the rod cells are many orders of magnitude less sensitive than at the stimulus wavelength, whereby the imaging lighting is not likely to disturb any effects caused by the stimulus.

In an embodiment of the invention, lighting for imaging of the pupil is performed at a wavelength where the rhodopsin sensitivity is at least one order of magnitude lower than at the stimulus wavelength.

In a further embodiment of the invention, lighting for imaging of the pupil is performed at a wavelength and intensity where the isomerization rate is at least one order of magnitude less than at the stimulus intensity and wavelength.

In a still further embodiment of the invention, lighting for imaging of the pupil is performed at a wavelength and intensity where the isomerization rate is at least two orders of magnitude less than with the stimulus intensity and wavelength.

In an embodiment of the invention, the stimuli are given at an intensity where the majority of resulting neural signals travel through the primary, most sensitive neural pathway, the rod bipolar pathway.

Which neural pathways the retinal signals take depends strongly on the intensity of light i.e. the isomerization rate. At 1 or less R* per rod per second neural signals travel almost completely through the primary rod bipolar pathway. Above a range of roughly 2 to 10 R* per rod per second the neural signals begin travelling through secondary rod pathways, as described e.g. in Grimes et al. (2018), Range, routing and kinetics of rod signaling in primate retina, eLife 7:e3828. At around 100 R* per rod per second most of the signals travel through secondary rod pathways. At around 1000 R* per rod per second or above virtually all of the signals travel through secondary rod pathways and cone photoreceptor-driven retinal pathways as well as melanopsin driven signaling (a light sensitive pigment in some of the retinal ganglion cell types), whereby measurements of pupillary responses do not give any information related to the primary rod bipolar pathway any more.

In an embodiment of the invention, the stimulus are given at an intensity resulting in an isomerization rate less than a first threshold, the first threshold being 1 R* per rod per second.

In an embodiment of the invention, the first threshold is 10 R* per rod per second.

In an embodiment of the invention, the first threshold is 100 R* per rod per second.

A man skilled in the art can calculate isomerization rates, i.e. number of isomerizations per rod caused by a light stimulus of a given wavelength or spectrum and intensity. In the following, we give examples of such calculations.

A man skilled in the art can measure the density of radiation (intensity) and spectral composition of light produced on a given surface, known as the irradiance spectrum (I(λ)). The light source generating this irradiance spectrum can be a monochromatic light source, such as a single wavelength LED with a bandpass filter, or the light source can be comprised of a broad range of wavelengths of light, such as a broad-spectrum LED or a DLP projector. A man skilled in the art can measure the irradiance spectrum generated by a light source at the plane where the cornea of the eye of a human observer is located (I_{cornea}(λ)) by placing a spectrophotometer at such location and measuring the irradiance values provided by the spectrophotometer in the form of a spectrum of bins of equal wavelength. By applying Plank's equation to calculating the energy of a single photon of a given wavelength (E(λ)), the photon flux at the cornea of the observer (φ_{cornea}(λ)) can then be calculated following the equation below. Detailed procedures for the application of such calculations have been published in G. Wyszecki and W. S. Stiles (1966) “Color Science: Concepts and Methods, Quantitative Data and Formulae”, John Wiley and Sons, New York, and many others.

$${ϕ}_{cornea} ( λ ) = \frac{{I}_{cornea} ( λ )}{E ( λ )}$$

From the photon flux at the cornea, a man skilled in the art can determine the photon flux at the retina of the human observer (φᵣₑₜᵢₙₐ(λ)) by taking into account the surface area of the observer's pupil (Aₚᵤₚᵢₗ(λ)), the surface area of the retina illuminated by the stimulus (Aᵣₑₜᵢₙₐ(λ)), and the transmissivity of ocular media in humans (T_{media}(λ)) by implementing the following relation:

$${ϕ}_{retina} ( λ ) = {ϕ}_{cornea} ( λ ) \frac{{A}_{pupil} ( λ )}{{A}_{retina} ( λ )} {τ}_{media} ( λ )$$

In an embodiment of the invention, the transmissivity of ocular media in humans can be obtained from the measured values published in J. van der Kraats and D. van Norren (2007) “Optical density of the aging human ocular media in the visible and the UV”, Journal of the Optical Society of America. A, Optics, image science, and vision, 24 (7): 1842-1857. In a different embodiment of the invention, the transmissivity of ocular media in humans can be obtained from the measured values published in the chapter K. H. Ruddock (1972) “Light Transmission through the Ocular Media and Macular Pigment and its Significance for Psychophysical Investigation” from the book “Visual Psychophysics”, Springer. This value includes all photon losses that occur as photons traverse the eye from the corneal surface to the surface of the retina.

From the photon flux at the surface of the retina, a man skilled in the art can determine the isomerization rate per rod photoreceptor cell by taking into consideration the collecting area of a single rod photoreceptor (Ac(λ)) and integrating over a wavelength interval over which the radiant flow of photons is emitted.

$${R}^{*} = \int {ϕ}_{retina} ( λ ) Ac ( λ ) d λ$$

In an embodiment of the invention, the collecting area of a single rod photoreceptor is calculated following the general procedure described in M. Kilpeläinen and others (2024) “Primate retina trades single-photon detection for high-fidelity contrast encoding”, Nature Communications, 15:4501 following the equation:

$$Ac ( λ ) = {π ( \frac{d}{2} )}^{2} ( 1 - 1 {0}^{D ( λ ) L} ) γ$$

Where d is the diameter of the rod outer segment where rhodopsin is located, L is the length of the rod outer segment, D(λ) specific axial absorbance of rhodopsin as a function of wavelength, and γ is the quantum efficiency of rhodopsin, yielding a value of 2.28 μm² for monochromatic light of 500 nm and 1.1*10⁻¹⁰ μm² for 940 nm.

In a further embodiment of the invention, the collecting area of a single rod photoreceptor is calculated following the general procedure described in A. Lyubarsky and E. N. Pugh (2004) “From candelas to photoisomerizations in the mouse eye by rhodopsin bleaching in situ and the light-rearing dependence of the major components of the mouse ERG”, Vision Research 44 3235-3251, with particular values adjusted to match those of the human retina.

In a further embodiment of the invention, the collecting area of a single rod photoreceptor is calculated following the general procedure described in D. A. Baylor and A. L. Hodgkin (1973) in their article “Detection and resolution of visual stimuli by turtle photoreceptors”, the Journal of Physiology, 234: 163-198 with particular values adjusted to match those of the human retina.

In the following, we provide some examples of isomerization rates produced by certain irradiance levels at the location of the cornea of the observer's eye, as calculated using the equations described previously. In an embodiment of the invention, an LED with a bandpass filter centered at 505 nm is utilized to deliver light stimuli homogeneously to the complete surface of the retina by means of a spherical chamber as illustrated in FIG. 1. Four different drive current settings producing increasingly higher load currents in the LED are tested. For each setting, the irradiance at the location of the cornea of the observer's eye (I_{cornea}(λ)) is measured yielding the values 1.1*10⁻⁹ W m⁻², 1.1*10⁻⁷ W m⁻², 1.1*10⁻⁵ W m⁻², and 1.1*10⁻³ W m⁻². By applying Plank's equation, a man skilled in the art can calculate the values of the photon flux at the cornea to be 2.88*10⁹ quanta s⁻¹ m⁻², 2.88* 10¹¹ quanta s⁻¹ m⁻², 2.88*10¹³ quanta s⁻¹ m⁻², 2.88*10¹⁵ quanta s⁻¹ m⁻². In this example, the area of the pupil of an observer's eye is 3.84*10⁻⁵ m², the area of the surface of the retina is assumed to be 1.094*10⁻³ m², and the transmission of the ocular media for 505 nm light is assumed to be 0.45. A man skilled in the art can then calculate the photon flux at the retinal surface to be 4.55*10⁷ quanta m⁻² s⁻¹, 4.55*10⁹ quanta m⁻² s⁻¹, 4.55*10¹¹ quanta m⁻² s⁻¹, 4.55*10¹³ quanta m⁻² s⁻¹; and the isomerization rate of rods to be 1*10⁻⁴, 1*10⁻², 1 and 100 R* rod⁻¹ s⁻¹ respectively.

Similarly, a man skilled in the art can calculate how much light is required to produce 1*10⁻³ and 1 R* rod⁻¹ s⁻¹ using monochromatic light of 940 nm in identical conditions as those described above. Considering a collecting area of 1.1*10⁻¹⁰ μm² for 940 nm, a photon flux at the surface of the retina of 9.09*10¹⁸ quanta m⁻² s⁻¹ and 9.09*10²¹ quanta m⁻² s⁻¹ would be required, corresponding to 5.75*10²⁰ quanta m⁻² s⁻¹ and 5.75*10²³ quanta m⁻² s⁻¹ at the surface of the cornea. Given the energy of a 940 nm photon to be 1.6*10⁻¹⁹ W s, the irradiance at the cornea required to produce 1*10⁻³ and 1 R* per rod s⁻¹ would then be 92 W m⁻² and 9.2*10⁴ W m⁻², respectively.

In an embodiment of the invention, the stimuli are given using light with wavelengths between 400 nm and 600 nm and at an intensity resulting in an irradiance at the location of the cornea of the observer's eye (I_{cornea}(λ)) of 1.1*10⁻⁹ W m⁻², or lower.

In an embodiment of the invention, the stimuli are given using light with wavelengths between 400 nm and 600 nm and at an intensity resulting in an irradiance at the location of the cornea of the observer's eye (I_{cornea}(λ)) of 1.1*10⁻⁷ W m⁻², or lower.

In an embodiment of the invention, the stimuli are given using light with wavelengths between 400 nm and 600 nm and at an intensity resulting in an irradiance at the location of the cornea of the observer's eye (I_{cornea}(λ)) of 1.1*10⁻⁵ W m⁻², or lower. In an embodiment of the invention, the stimuli are given using light with wavelengths between 400 nm and 600 nm and at an intensity resulting in an irradiance at the location of the cornea of the observer's eye (I_{cornea}(λ)) of 1.1*10⁻³ W m⁻², or lower.

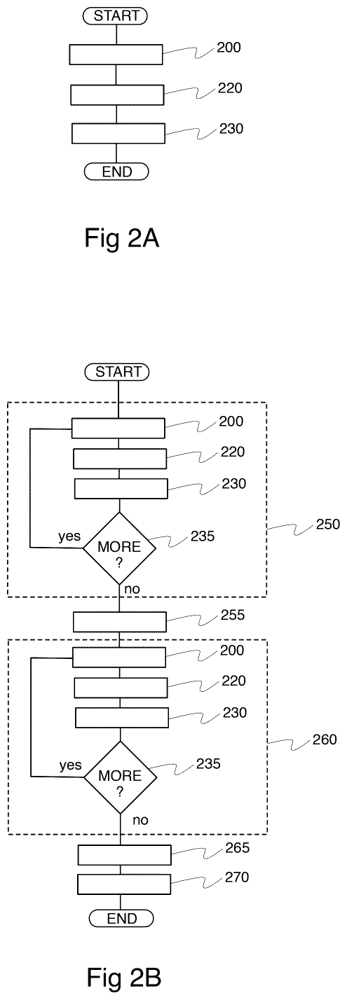

FIG. 2A illustrates a method for measuring a characteristic of mammalian retina according to an embodiment of the invention. In the method stimulus is provided to the eye using a light source and responses of the eye to the provided stimulus are monitored. In an embodiment of the invention the method comprises at least the steps of
providing 200 stimulus using light within a first wavelength range at an intensity causing an isomerization rate of less than 100 R* per rod per second;
monitoring 220 responses of the eye using a camera sensitive to light in the second wavelength range for imaging the eye and an imaging light source for illuminating the eye, where said imaging light source is arranged to provide light within the second wavelength range and at an intensity causing a lower isomerization rate than said light stimulus, and
monitoring responses of the eye by determining 230 the size of the pupil in images provided by said camera.

FIG. 2B illustrates a method for measuring a characteristic of mammalian retina according to an embodiment of the invention. In this embodiment, the method further comprises at least the steps of
measuring 250 pupillary responses to negative stimulus,
determining 255 the smallest negative stimulus level that results in a detectable pupillary response,
measuring 260 pupillary responses to positive stimulus,
determining 265 the smallest positive stimulus level that results in a detectable pupillary response, and
determining 270 a characteristic of the retina based on said smallest negative stimulus level and/or said smallest positive stimulus level.

In step 250 of measuring pupillary responses to negative stimuli the steps of providing stimuli, monitoring responses of the eye, and determining the size of the pupil may be repeated multiple times while changing the stimuli. Similarly, in step 260 of measuring pupillary responses to positive stimuli, monitoring responses of the eye, and determining the size of the pupil may be repeated multiple times while changing the stimuli. These steps can in various embodiments be repeated for example for a predetermined number of times, or until a predetermined condition is fulfilled. The predetermined condition can be for example detection of a pupillary response as a result of increasing stimulus, or for example observation of cessation of pupillary response as a response to lessening stimulus. The decision of whether or not to repeat is represented by item 235 in FIG. 2B.

Further, in an embodiment of the invention the steps 250 to 265 may be ordered otherwise. For example, measuring pupillary responses to positive stimulus may be performed before the step of measuring pupillary responses to negative stimulus.

FIG. 2B illustrates an embodiment where responses to both negative and positive stimuli are measured. However, in a further embodiment of the invention, only responses to negative stimuli are measured. In an even further embodiment of the invention, only responses to positive stimuli are measured.

In a further embodiment of the invention, in the step of monitoring responses of the eye by determining the size of the pupil, changes in size of the pupil within a timescale of 0 to 30 seconds after a stimulus are monitored. In a still further embodiment of the invention, in the step of monitoring responses of the eye by determining the size of the pupil, changes in size of the pupil within a timescale of 0 to 3 seconds after a stimulus are monitored.

The inventive system and method can be used to examine retinal characteristics in various mammals, such as humans, dogs, monkeys and mice.

### Certain Benefits of the Invention

The invention has many benefits. The invention provides a noninvasive method for determining retinal characteristics. The invention provides a way to examine most sensitive rod bipolar pathways of the retina, which are sensitive to various types of damage. The invention provides a system and method that automates a large part of the examination, thus considerably shortening the time required for such an examination. Further, the invention provides a system and method that does not require instruction and training of a human subject of examination.

### Certain Further Observations

In view of the foregoing description it will be evident to a person skilled in the art that various modifications may be made within the scope of the invention. While a preferred embodiment of the invention has been described in detail, it should be apparent that many modifications and variations thereto are possible, all of which fall within the true spirit and scope of the invention.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the previous description, numerous specific details are provided, such as examples of lengths, widths, shapes, etc., to provide a thorough understanding of embodiments of the invention.

One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A system (100) for determining a characteristic of mammalian retina having a first light source for providing stimulus to the eye and an arrangement for monitoring responses of the eye to the stimulus, characterized in that
the first light source is arranged to provide stimulus using light within a first wavelength range and at an intensity causing on average a lower isomerization rate than a first threshold, the average being calculated over at least one stimulus cycle;
said first threshold being 100 R* per rod per second,
the arrangement for monitoring responses of the eye comprises at least a camera for imaging the eye at a second wavelength range and an imaging light source for illuminating the eye at said second wavelength range;
said imaging light source being arranged to provide light at an intensity causing isomerizations at a lower rate than said stimulus with light within said first wavelength range,
said system further comprising a control unit arranged to control the light source providing stimuli to the eye,
receive images from said camera, and
determine response of the pupil to the stimulus from said received images.

2. A system according to claim 1, characterized in that
said first threshold is 10 R* per rod per second.

3. A system according to claim 1, characterized in that
said first threshold is 1 R* per rod per second.

4. A system according to claim 1, characterized in that
the first wavelength range is from 300 to 900 nanometers.

5. A system according to claim 1, characterized in that
the first wavelength range is from 400 to 600 nanometers.

6. A system according to claim 1, characterized in that
said second wavelength range is wavelengths of 700 nm or longer.

7. A system according to claim 1, characterized in that
said control unit is arranged to monitor changes in the pupil within a timescale of 0 to 30 seconds after a stimulus.

8. A system according to claim 1, characterized in that the system is arranged to measure pupillary responses to negative stimulus,
determine the smallest negative stimulus level that results in a detectable pupillary response, and
determine a characteristic of the retina at least in part based on said smallest negative stimulus level.

9. A system according to claim 1, characterized in that the system is arranged to measure pupillary responses to positive stimulus,
determine the smallest positive stimulus level that results in a detectable pupillary response, and
determine a characteristic of the retina at least in part based on said smallest positive stimulus level.

10. A method for measuring of a characteristic of mammalian retina, in which stimulus is provided to the eye using a light source and responses of the eye to the provided stimulus are monitored, characterized in that the method comprises at least the steps of
providing stimulus using light within a first wavelength range at an intensity causing an isomerization rate lower than a first threshold, said first threshold being 100 R* per rod per second;
monitoring responses of the eye using a camera sensitive to wavelength in a second wavelength range for imaging the eye and an imaging light source for illuminating the eye, where said imaging light source is arranged to provide light within said second wavelength range and at an intensity causing isomerizations at a lower rate than said light stimulus, and
monitoring responses of the eye by determining the size of the pupil in images provided by said camera.

11. A method according to claim 10, characterized in that
said first threshold is 10 R* per rod per second.

12. A method according to claim 10, characterized in that
said first threshold is 1 R* per rod per second.

13. A method according to claim 10, characterized in that
in the step of monitoring responses of the eye by determining the size of the pupil, changes in size of the pupil within a timescale of 0 to 30 seconds after a stimulus are monitored.

14. A method according to claim 10, characterized in that the method further comprises at least the steps of measuring pupillary responses to negative stimulus,
determining the smallest negative stimulus level that results in a detectable pupillary response, and
determining a characteristic of the retina at least in part based on said smallest negative stimulus level.

15. A method according to claim 10, characterized in that the method further comprises at least the steps of measuring pupillary responses to positive stimulus,
determining the smallest positive stimulus level that results in a detectable pupillary response, and
determining a characteristic of the retina at least in part based on said smallest positive stimulus level.
